(19) United States
(12) Patent Application Publication
(10) Pub. No.: US 2022/0375556 A1
(43) Pub. Date: Nov. 24, 2022

(54) METHOD AND SYSTEM FOR ELECTRONIC MEDICAL RECORD CREATION AND MEDICAL BILLING

(71) Applicant: Codified Health Incorporated, Rancho Palos Verdes, CA (US)

(72) Inventors: Sophia Ghauri, Houston, TX (US); Hailey Chen, Rancho Palos Verdes, CA (US); Cindy Zheng, Baton Rouge, LA (US); Omar Imtiza, Houston, TX (US)

(21) Appl. No.: 17/749,819

(22) Filed: May 20, 2022

Related U.S. Application Data
(60) Provisional application No. 63/191,329, filed on May 21, 2021.

(51) Int. Cl. G16H 10/60

(52) U.S. Cl. CPC G16H 10/60

(57) ABSTRACT

A computer-implemented method for medical billing includes providing a list of medical billing codes comprising standardized statistical codes, reading physician notes pertaining to a physician-patient meeting, executing a tokenization process on the physician notes, thereby producing a plurality of tokens from the physician notes, executing a natural language processing transformer comprising a dense, deep neural network trained on a medical language corpus, wherein the transformer is configured for reading and processing the plurality of tokens, and producing a plurality of output items from the plurality of tokens, wherein each output item comprises a clinical statement, for each of the plurality of output items, finding a matching medical billing code from the list of medical billing codes, thereby producing a plurality of medical billing codes, and storing said medical billing codes in an electronic health record in association with the physician notes.

### CROSS-REFERENCE TO RELATED APPLICATIONS

This patent application claims priority to provisional patent application No. 63/191,329 filed on May 21, 2021 and titled “Electronic Health Records System Utilizing AI for Physician and Patient Chart Creation and Electronic Billing.” The contents of provisional patent application No. 63/191,329 are hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The claimed subject matter relates to the field of medicine and, more specifically, the claimed subject matter relates to the field of patient chart creation and medical billing.

### BACKGROUND

The terms patient chart, medical record, health record and medical chart refer to the systematic documentation of a patient's medical history and care across time. A medical record includes a variety of types of notes entered over time by healthcare professionals, recording observations and administration of drugs and therapies, orders for the administration of drugs and therapies, test results, x-rays, reports, etc. The maintenance of complete and accurate medical records is a requirement of health care providers and is generally enforced as a licensing or certification prerequisite. Medical records have traditionally been compiled and maintained by health care providers, and advances in technology have led to the development of electronic health records (EHR), which is the systematized collection of patient and population electronically stored health information in a digital format. EHRs can be shared across different health care settings and can be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information. An EHR is usually created and/or edited when a health-related event occurs, such as a doctor's visit, a test result that has become available, or data that has been collected.

Medical billing is a payment practice within the United States health system. The process involves a healthcare provider obtaining insurance information from a patient, filing a claim, following up on, and appealing claims with health insurance companies in order to receive payment for services rendered; such as testing, treatments, and procedures. The same process is used for most insurance companies, whether they are private companies or government sponsored programs. Namely, medical coding reports what the diagnosis and treatment were, and prices are applied accordingly. Medical coding refers to the medical classification used to transform descriptions of medical diagnoses or procedures into standardized statistical codes in a process known as clinical coding. Diagnosis classifications list diagnosis codes, which are used to track diseases and other health conditions, inclusive of chronic diseases such as diabetes mellitus and heart disease, and infectious diseases such as norovirus, the flu, and athlete's foot. Procedure classifications list procedure code, which are used to capture interventional data. These diagnosis and procedure codes are used by health care providers, government health programs, private health insurance companies, workers' compensation carriers, software developers, and others for a variety of applications.

One of the drawbacks associated with conventional EHR and medical billing activities involves the time and energy necessary to convert physician notes into proper notes for inclusion in an EHR and into proper medical billing codes for medical billing. Conventionally, a medical professional must devote time to convert said physician notes for inclusion in an EHR and into proper medical billing codes for medical billing. This can be time-consuming and tedious for medical professionals. Further, some medical professional lack the proper training to convert physician notes into proper notes for inclusion in an EHR and into proper medical billing codes. Additionally, said conversion of physician notes into medical billing codes are subject to user error, which increase costs and billing cycle time. This can be disadvantageous, especially when involving medical entities that are already overtaxed and have very narrow margins of profitability.

Therefore, what is needed is a system and method for improving the problems with the prior art, and more particularly for a more expedient and efficient method and system for facilitating EHR and medical billing activities.

### BRIEF SUMMARY

In one embodiment, a computer-implemented method for medical billing is disclosed. The method includes providing a list of medical billing codes comprising standardized statistical codes, reading physician notes pertaining to a physician-patient meeting, executing a tokenization process on the physician notes, thereby producing a plurality of tokens from the physician notes, executing a natural language processing transformer comprising a dense, deep neural network trained on a medical language corpus, wherein the transformer is configured for reading and processing the plurality of tokens, and producing a plurality of output items from the plurality of tokens, wherein each output item comprises a clinical statement, for each of the plurality of output items, finding a matching medical billing code from the list of medical billing codes, thereby producing a plurality of medical billing codes, and storing said medical billing codes in an electronic health record in association with the physician notes.

Additional aspects of the claimed subject matter will be set forth in part in the description which follows, and in part will be obvious from the description, or may be learned by practice of the claimed subject matter. The aspects of the claimed subject matter will be realized and attained by means of the elements and combinations particularly pointed out in the appended claims. It is to be understood that both the foregoing general description and the following detailed description are exemplary and explanatory only and are not restrictive of the disclosed subject matter, as claimed.

### BRIEF DESCRIPTION OF THE SEVERAL VIEWS OF THE DRAWINGS

The accompanying drawings, which are incorporated in and constitute part of this specification, illustrate embodiments of the claimed subject matter and together with the description, serve to explain the principles of the claimed subject matter. The embodiments illustrated herein are presently preferred, it being understood, however, that the claimed subject matter is not limited to the precise arrangements and instrumentalities shown, wherein:

FIG. 1 is a block diagram illustrating the network architecture of a system for medical billing over a communications network, in accordance with one embodiment.

FIG. 2A is a block diagram showing the data flow of the process for medical billing over a communications network, according to one embodiment.

FIG. 2B is a block diagram depicting the process for medical billing over a communications network, according to one embodiment.

FIG. 3 is a flow chart depicting the general control flow of a process for medical billing over a communications network, according to one embodiment.

FIG. 4 is a block diagram depicting a system including an example computing device and other computing devices.

### DETAILED DESCRIPTION

It should be understood that the embodiments disclosed herein are only examples of the many advantageous uses of the innovative teachings of the claimed embodiments. In general, statements made in the specification of the present application do not necessarily limit any of the various claimed embodiments. Moreover, some statements may apply to some inventive features but not to others. In general, unless otherwise indicated, singular elements may be in the plural and vice versa with no loss of generality. In the drawing like numerals refer to like parts through several views.

The disclosed embodiments improve upon the problems with the prior art by providing a system that allows for the fast, accurate and easy process of creating electronic health records (EHR) in an automated fashion from physician notes without requiring additional time or attention from medical professionals. Therefore, the disclosed embodiments reduce or eliminate the need for the medical professionals to take time out of their day to convert physician notes into proper clinical statements for inclusion in an EHR. This is advantageous for users, as it saves time and money. An additional benefit of the disclosed embodiments is quick, precise and simple process of generating medical billing codes in an automated fashion from physician notes without requiring additional work or input from medical professionals. Therefore, the disclosed embodiments reduce or eliminate the need for the medical professionals to dedicating more work hours to converting physician notes into proper medical billing codes for medical billing. This is advantageous as it also saves time and money.

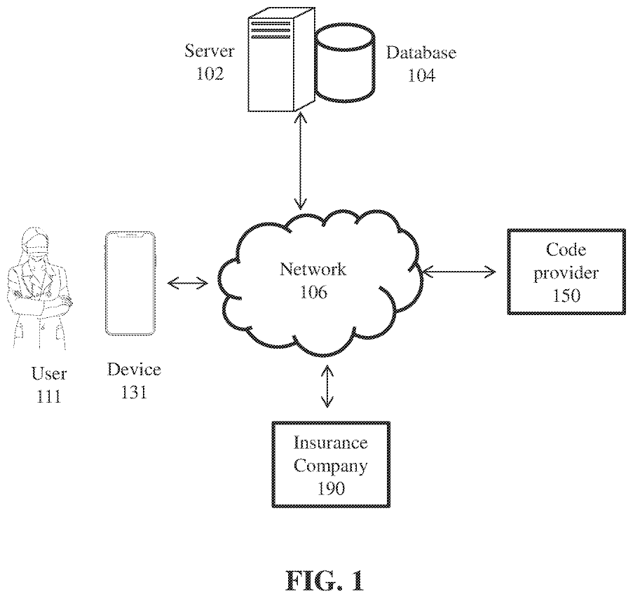

Referring now to the drawing figures in which like reference designators refer to like elements, there is shown in FIG. 1 an illustration of a block diagram showing the network architecture of a system 100 and method for facilitating medical billing activities over a communications network in accordance with one embodiment. A prominent element of FIG. 1 is the server 102 associated with repository or database 104 and further communicatively coupled with network 106, which can be a circuit switched network, such as the Public Service Telephone Network (PSTN), or a packet switched network, such as the Internet or the World Wide Web, the global telephone network, a cellular network, a mobile communications network, or any combination of the above. Server 102 is a central controller or operator for functionality of the disclosed embodiments, namely, facilitating gift giving activities between users.

FIG. 1 includes mobile computing device 131, which may be smart phones, mobile phones, tablet computers, handheld computers, laptops, or the like. In another embodiment, mobile computing device 131 may be workstations, desktop computers, servers, laptops, all-in-one computers, or the like. In another embodiment, mobile computing device 131 may be AR or VR systems that may include display screens, headsets, heads up displays, helmet mounted display screens, tracking devices, tracking lighthouses or the like. Mobile computing device 131 corresponds to a user 111 which may be a medical professional such as a doctor, a nurse, etc. Device 131 may be communicatively coupled with network 106 in a wired or wireless fashion. Augmented reality (AR) adds digital elements to a live view often by using a camera on a computing device. Virtual reality (VR) is a complete or near complete immersion experience that replaces the physical world.

FIG. 1 further shows that server 102 includes a database or repository 104, which may be a relational database comprising a Structured Query Language (SQL) database stored in a SQL server. Device 131 may also include its own database. The repository 104 serves data from a database, which is a repository for data used by server 102 and device 131 during the course of operation of the disclosed embodiments. Database 104 may be distributed over one or more nodes or locations that are connected via network 106.

The database 104 may include a user record for each user 111. A user record may include: contact/identifying information for the user (name, address, telephone number(s), email address, etc.), information pertaining to physician notes associated with the user, contact/identifying information for patients of the user, electronic payment information for the user, information pertaining to the medical billing codes used by the user, sales transaction data associated with the user, etc. A user record may also include a unique identifier for each user, a residential address for each user, the current location of each user (based on location-based services from the user's mobile computer) and a description of past doctor's visits associated with each user. A user record may further include demographic data for each user, such as age, sex, income data, race, color, marital status, etc.

The database 104 may include an EHR for each patient. An EHR may include a variety of types of notes entered over time by healthcare professionals, recording observations and administration of drugs and therapies, orders for the administration of drugs and therapies, test results, x-rays, reports, etc. An EHR may also include a range of data, including demographics, medical history, physician notes, clinical statements, medication and allergies, immunization status, laboratory test results, radiology images, vital signs, personal statistics like age and weight, and billing information. An EHR may also include medical billing codes corresponding to clinical statements made by a medical professional in the HER. A clinical statement may comprise a human language readable string, such as an English sentence, that corresponds to a medical billing code, such as the statement “administering a COVID-19 test.”

The database 104 may also be used to store medical billing codes used for the processes of the claimed embodiments. Medical billing codes may refer to standardized statistical codes using a classification system wherein said codes correspond to clinical statements made by a medical professional. Said codes may originate from a set of published codes on medical diagnoses and procedures, such as the International Classification of Diseases (ICD), the Healthcare Common procedural Coding System (HCPCS), and Current Procedural Terminology (CPT) for reporting to the health insurance provider 190 of the recipient of the care. The use of standardized statistical codes allows insurance providers to map equivalencies across different service providers who may use different terminologies or abbreviations in their written claims forms, and be used to justify reimbursement of fees and expenses. The codes may cover topics related to diagnoses, procedures, pharmaceuticals or topography. The standardized statistical codes may, in one embodiment, comprises a number or a string of numbers, wherein said number may be a real number. Said codes may originate from a medical code provider 150. FIG. 1 shows that that the entities 150, 190 may be connected to the network 106.

FIG. 1 shows an embodiment wherein networked computing devices 131, 150, 190 interact with server 102 and repository 104 over the network 106. It should be noted that although FIG. 1 shows only the networked computers 131, 150, 190 and 102, the system of the disclosed embodiments supports any number of networked computing devices connected via network 106. Further, server 102, and unit 131 include program logic such as computer programs, mobile applications, executable files or computer instructions (including computer source code, scripting language code or interpreted language code that may be compiled to produce an executable file or that may be interpreted at run-time) that perform various functions of the disclosed embodiments.

Note that although server 102 is shown as a single and independent entity, in one embodiment, the functions of server 102 may be integrated with another entity, such as one of the devices 131, 150, 190. Further, server 102 and its functionality, according to a preferred embodiment, can be realized in a centralized fashion in one computer system or in a distributed fashion wherein different elements are spread across several interconnected computer systems. Note also that although FIG. 1 only shows a single device 131, the claimed embodiments support any number of devices and users connected via network 106.

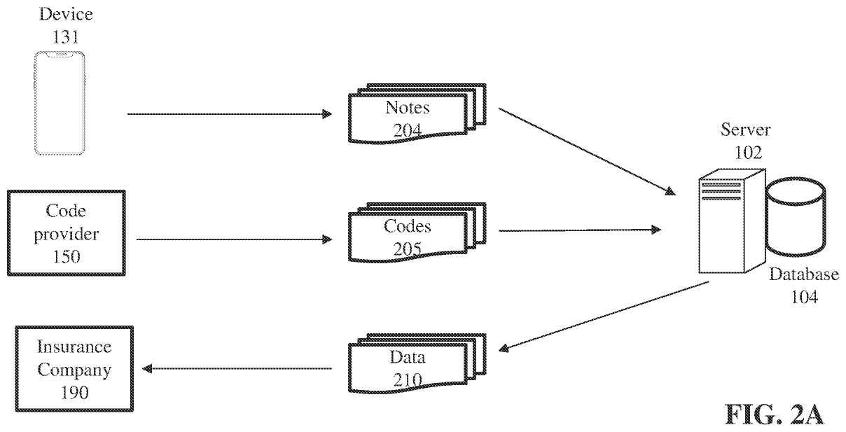

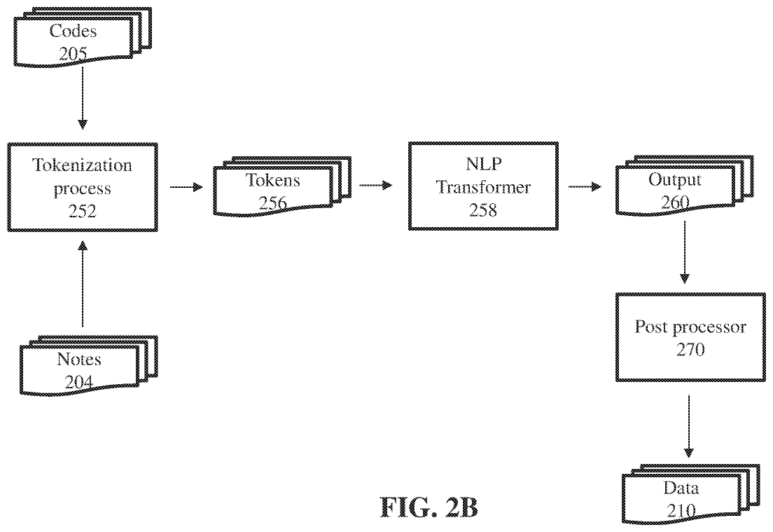

FIG. 2B depicts a block diagram of the process for facilitating medical billing over a communications network 106, according to one embodiment. FIG. 2B shows a tokenization process 252. Tokenization is the well-known process of demarcating and classifying sections of a string of input characters. The resulting tokens are then passed on to some other form of processing. The tokenization process can be considered a sub-task of parsing input. Inputs to the tokenization process are tokenized and padded into embeddings as follows: inputs are split into token embeddings, one per token, segment embeddings are found for each token, and positional embeddings are found for each token. Subsequently, each embedding is added together to produce the output of the tokenization process, which is a plurality of tokens. A token may be a text string comprising human readable language such as a sentence.

FIG. 2B also shows a natural language processing (NLP) transformer process 258. A NLP transformer process is a deep learning model that adopts the mechanism of self-attention, differentially weighting the significance of each part of the input data. Like a recurrent neural network (RNN), a NLP transformer is designed to process sequential input data, i.e., natural language, for the production of clinical statements. Unlike an RNN, the NLP transformer processes the entire input all at once. The attention mechanism provides context for any position in the input sequence.

The transformer process 258 is a dense, deep neural network trained on a medical English language corpus. Blocks of medical English language text are used for training the NLP transformer process 258. In each iteration of training, a token from the block is removed, and the network's target is to predict the missing token. Fine-tuning training is also performed on a medical English language corpus wherein approximately 1% of training time is spent on domain-specific training.

In one embodiment, the transformer process 258 is a bi-directional encoder representations from transformers process, which is a transformer-based machine learning technique for NLP pre-training. In this case, it is a dense, deep neural network that reads entire sequences of data (i.e., the tokens produced by the tokenization process) simultaneously to gain bi-directional context. It also contains classification and response layers in the output for different use-cases and training.

FIG. 2B also shows a post processor 270 configured for reading the plurality of output items (clinical statements) produced by the transformer process 258, finding a matching medical billing code from the list of medical billing codes, and producing a plurality of medical billing codes. In one embodiment, the list of medical billing codes is stored in an array or a linked list. Associated with each medical billing code in the array or linked list is one or more clinical statements. In one embodiment, each medical billing code in the array or linked list is associated with a pointer that points to an array or linked list that holds the one or more clinical statements associated with that medical billing code. Said arrangement of a list of medical billing codes stored in an array or a linked list wherein each medical billing code points to an array or linked list of clinical statements is referred to herein as a data structure.

In this embodiment, the process of finding a matching medical billing code for a clinical statement comprises looking up the clinical statement in the data structure. Once the clinical statement is found in the data structure, the post processor 270 finds the medical billing code associated with that clinical statement in the data structure, i.e., the medical billing code in the array or linked list that points to the clinical statement that was found.

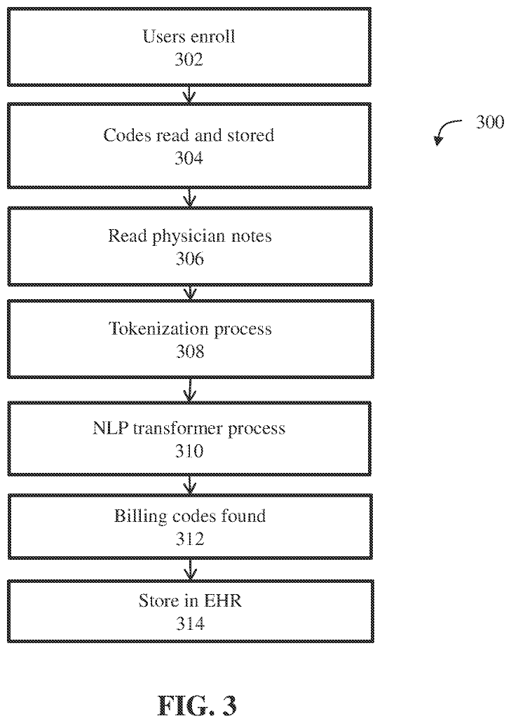

The process of medical billing over a communications network will now be described with reference to FIGS. 2A, 2B, 3 below. FIGS. 2A and 3 depict the data flow and control flow of the process for facilitating medical billing over a communications network 106, according to one embodiment. The process of the disclosed embodiments begins with optional step 302 (see flowchart 300), wherein the user 111 may enroll or register with server 102. In the course of enrolling or registering, the user may enter data into their device by manually entering data into a mobile application via keypad, touchpad, or via voice. In the course of enrolling or registering, the user may enter any data that may be stored in a user record, as defined above. Also in the course of enrolling or registering, the server 102 may generate a user record for each registering user and store the user record in an attached database, such as database 104.

Subsequently, in step 304, medical billing codes 205 may be stored by database 104. In one embodiment, said codes 205 are read from, or uploaded by, the code provider 150 via network 106. The medical billing codes may comprise standardized statistical codes.

In step 306, physician notes 204 may be stored by database 104. In one embodiment, said physician notes 204 are read from, or uploaded by, the device 131 of medical professional 111 via network 106. The physician notes may pertain to a physician-patient meeting. The physician notes may be a text string.

In the next step 308, the tokenization process 252 operating on server 102 is executed. Said process 252 takes as input the codes 205 and notes 204. The process 252 produces a plurality of tokens 256 from the physician notes.

In the next step 310, the NLP transformer process 258 operating on server 102 is executed. Said process 258 takes as input the plurality of tokens 256. The process 258 produces a plurality of output items 260. The NLP transformer may comprise a dense, deep neural network trained on a medical language corpus, wherein the transformer is configured for reading and processing the plurality of tokens 256, and producing a plurality of output items 260, wherein each output item comprises a clinical statement.

In the next step 312, the post processor 270 operating on server 102 is executed. Said post processor 270 takes as input the plurality of output items 260. The post processor 270 produces a plurality of data 210 which may include a plurality of medical billing codes. In step 314, said data 210 is stored in the EHR of the patient. In an optional step, the medical billing codes of data 210 are transmitted via network 106 in a medical bill to the insurance company 190 for payment.

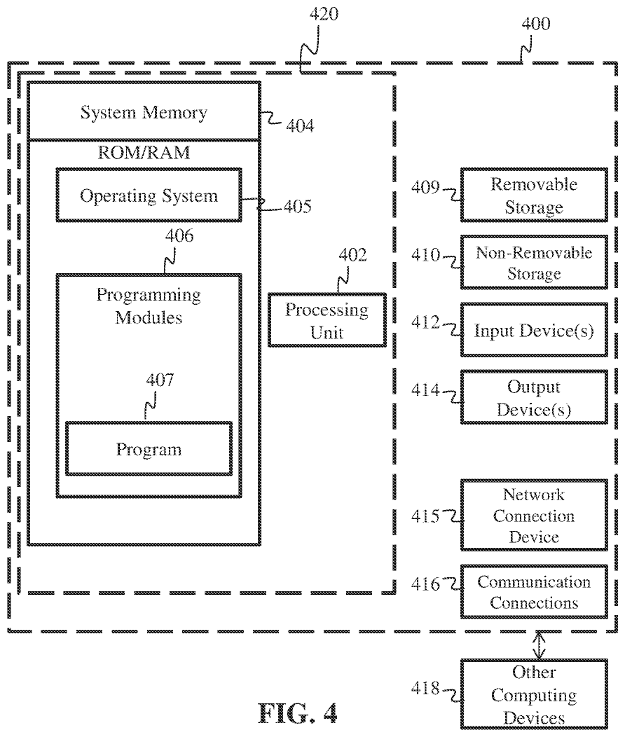

FIG. 4 is a block diagram of a system including an example computing device 400 and other computing devices. Consistent with the embodiments described herein, the aforementioned actions performed by 131, 102 may be implemented in a computing device, such as the computing device 400 of FIG. 4. Any suitable combination of hardware, software, or firmware may be used to implement the computing device 400. The aforementioned system, device, and processors are examples and other systems, devices, and processors may comprise the aforementioned computing device. Furthermore, computing device 400 may comprise an operating environment for system 100 and process 300, as described above. Process 300 may operate in other environments and are not limited to computing device 400.

With reference to FIG. 4, a system consistent with an embodiment may include a plurality of computing devices, such as computing device 400. In a basic configuration, computing device 400 may include at least one processing unit 402 and a system memory 404. Depending on the configuration and type of computing device, system memory 404 may comprise, but is not limited to, volatile (e.g. random-access memory (RAM)), non-volatile (e.g. read-only memory (ROM)), flash memory, or any combination or memory. System memory 404 may include operating system 405, and one or more programming modules 406. Operating system 405, for example, may be suitable for controlling computing device 400's operation. In one embodiment, programming modules 406 may include, for example, a program module 407 for executing the actions of 131, 102. Furthermore, embodiments may be practiced in conjunction with a graphics library, other operating systems, or any other application program and is not limited to any particular application or system. This basic configuration is illustrated in FIG. 4 by those components within a dashed line 420.

Computing device 400 may have additional features or functionality. For example, computing device 400 may also include additional data storage devices (removable and/or non-removable) such as, for example, magnetic disks, optical disks, or tape. Such additional storage is illustrated in FIG. 4 by a removable storage 409 and a non-removable storage 410. Computer storage media may include volatile and nonvolatile, removable and non-removable media implemented in any method or technology for storage of information, such as computer readable instructions, data structures, program modules, or other data. System memory 404, removable storage 409, and non-removable storage 410 are all computer storage media examples (i.e. memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 400. Any such computer storage media may be part of device 400. Computing device 400 may also have input device(s) 412 such as a keyboard, a mouse, a pen, a sound input device, a camera, a touch input device, etc. Output device(s) 414 such as a display, speakers, a printer, etc. may also be included. Computing device 400 may also include a vibration device capable of initiating a vibration in the device on command, such as a mechanical vibrator or a vibrating alert motor. The aforementioned devices are only examples, and other devices may be added or substituted.

Computing device 400 may also contain a network connection device 415 that may allow device 400 to communicate with other computing devices 418, such as over a network in a distributed computing environment, for example, an intranet or the Internet. Device 415 may be a wired or wireless network interface controller, a network interface card, a network interface device, a network adapter or a LAN adapter. Device 415 allows for a communication connection 416 for communicating with other computing devices 418. Communication connection 416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 404, including operating system 405. While executing on processing unit 402, programming modules 406 (e.g. program module 407) may perform processes including, for example, one or more of the stages of the process 300 as described above. The aforementioned processes are examples, and processing unit 402 may perform other processes. Other programming modules that may be used in accordance with embodiments herein may include electronic mail and contacts applications, word processing applications, spreadsheet applications, database applications, slide presentation applications, drawing or computer-aided application programs, etc.

Generally, consistent with embodiments herein, program modules may include routines, programs, components, data structures, and other types of structures that may perform particular tasks or that may implement particular abstract data types. Moreover, embodiments herein may be practiced with other computer system configurations, including hand-held devices, multiprocessor systems, microprocessor-based or programmable consumer electronics, minicomputers, mainframe computers, and the like. Embodiments herein may also be practiced in distributed computing environments where tasks are performed by remote processing devices that are linked through a communications network. In a distributed computing environment, program modules may be located in both local and remote memory storage devices.

Furthermore, embodiments herein may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments herein may also be practiced using other technologies capable of performing logical operations such as, for example, AND, OR, and NOT, including but not limited to mechanical, optical, fluidic, and quantum technologies. In addition, embodiments herein may be practiced within a general purpose computer or in any other circuits or systems.

Embodiments herein, for example, are described above with reference to block diagrams and/or operational illustrations of methods, systems, and computer program products according to said embodiments. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments have been described, other embodiments may exist. Furthermore, although embodiments herein have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the claimed subject matter.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A computer-implemented method for medical billing, the method comprising the steps of:
a) providing a list of medical billing codes comprising standardized statistical codes;
b) reading physician notes pertaining to a physician-patient meeting;
c) executing a tokenization process on the physician notes, thereby producing a plurality of tokens from the physician notes;
d) executing a natural language processing transformer comprising a dense, deep neural network trained on a medical language corpus, wherein the transformer is configured for reading and processing the plurality of tokens, and producing a plurality of output items from the plurality of tokens, wherein each output item comprises a clinical statement;
e) for each of the plurality of output items, finding a matching medical billing code from the list of medical billing codes, thereby producing a plurality of medical billing codes; and
f) storing said medical billing codes in an electronic health record in association with the physician notes.

2. The method of claim 1, wherein a standardized statistical code comprises a number.

3. The method of claim 2, wherein a physician note comprises a text string.

4. The method of claim 3, wherein a token comprises a text string.

5. The method of claim 4, wherein a clinical statement comprises a text string.

6. A non-transitory computer-readable medium with instructions stored thereon, that when executed by a processor, perform the steps comprising:
a) providing a list of medical billing codes comprising standardized statistical codes;
b) reading physician notes pertaining to a physician-patient meeting;
c) executing a tokenization process on the physician notes, thereby producing a plurality of tokens from the physician notes;
d) executing a natural language processing transformer comprising a dense, deep neural network trained on a medical language corpus, wherein the transformer is configured for reading and processing the plurality of tokens, and producing a plurality of output items from the plurality of tokens, wherein each output item comprises a clinical statement;
e) for each of the plurality of output items, finding a matching medical billing code from the list of medical billing codes, thereby producing a plurality of medical billing codes; and
f) storing said medical billing codes in an electronic health record in association with the physician notes.

7. The non-transitory computer-readable medium of claim 6, wherein a standardized statistical code comprises a number.

8. The non-transitory computer-readable medium of claim 7, wherein a physician note comprises a text string.

9. The non-transitory computer-readable medium of claim 8, wherein a token comprises a text string.

10. The non-transitory computer-readable medium of claim 9, wherein a clinical statement comprises a text string.

11. A system for medical billing, comprising a processor configured for:
a) providing a list of medical billing codes comprising standardized statistical codes;
b) reading physician notes pertaining to a physician-patient meeting;
c) executing a tokenization process on the physician notes, thereby producing a plurality of tokens from the physician notes;
d) executing a natural language processing transformer comprising a dense, deep neural network trained on a medical language corpus, wherein the transformer is configured for reading and processing the plurality of tokens, and producing a plurality of output items from the plurality of tokens, wherein each output item comprises a clinical statement;
e) for each of the plurality of output items, finding a matching medical billing code from the list of medical billing codes, thereby producing a plurality of medical billing codes; and
f) storing said medical billing codes in an electronic health record in association with the physician notes.

12. The system of claim 11, wherein a standardized statistical code comprises a number.

13. The system of claim 12, wherein a physician note comprises a text string.

14. The system of claim 13, wherein a token comprises a text string.

15. The system of claim 14, wherein a clinical statement comprises a text string.
